(19) United States
(12) Patent Application Publication
(10) Pub. No.: US 2016/0166842 A1
(43) Pub. Date: Jun. 16, 2016

(54) MONITOR DEFIBRILLATOR WITH MULTI-ANGLE POSITIONING

(71) Applicant: KONINKLIJKE PHILIPS N.V., EINDHOVEN (NL)

(72) Inventors: PATRICK GUINEY, CONCORD, MA (US); STEPHEN ROBERT HEATH, ANDOVER, MA (US); JOHN BENCH CASWELL, MERRIMACK, NH (US)

(21) Appl. No.: 14/965,065

(22) Filed: Dec. 10, 2015

Related U.S. Application Data
(60) Provisional application No. 62/090,049, filed on Dec. 10, 2014.

(51) Int. Cl. A61N 1/39; G06F 3/041

(52) U.S. Cl. CPC A61N 1/3968; G06F 3/0412; A61N 1/3993

(57) ABSTRACT

An emergency medical device includes a treatment device including a display face, a base including a bottom portion configured to rest on a resting surface, and a back portion configured to extend beyond a rear surface of the treatment device opposite the front portion. A rear structure is connected to the treatment device and extends beyond the rear surface of the treatment device wherein the base is configured to provide a first angle of the display face when the bottom portion is resting on the resting surface, and wherein the back portion and the rear structure provide a second angle of the display face when the back portion and the rear structure are resting on the resting surface.

### BACKGROUND

1. Technical Field

This disclosure relates to medical instruments and more particularly to a treatment device with a touch screen including multiple pre-set angle positions for usage in different positions.

2. Description of the Related Art

Heart monitors and/or defibrillators typically include mechanical control knobs buttons and switches. With the current generation of products, the user directly controls power and adjusts settings by manually turning the device on and off, and adjusting knobs and buttons to achieve desired settings. Newer defibrillator/monitor devices include advanced features and LCD displays but lack the sophistication and flexibility of modern interfaces, such as touchscreens, or other advanced devices.

Monitor/defibrillator screens are typically oriented at 60° to 90° relative to the horizontal plane or resting surface. Medics arriving at accident scenes where patients collapse or are otherwise on the ground must position the monitor defibrillator next to patient on the floor or ground. While treating the patient from a kneeling position, medics must also operate and manipulate the monitor/defibrillator.

Operating a device positioned at 60° to 90° relative to the floor or ground when kneeling on the floor or ground requires an awkward wrist posture. When using a touchscreen, the operator must visualize the display while positioning their hand below the target touch zone and then bend the wrist upward to touch the desired button. The hazards of increasing the wrist angle of bending are well documented in medical literature. Repetitive, upward bending of the wrist causes irritation of tendons and tendon sheaths and will lead to a variety of health consequences including carpal tunnel syndrome. The angle of 60° to 90° relative to a horizontal surface is more appropriate for operating the monitor/defibrillator from a seated or standing posture when transporting a patient in an ambulance or on a gurney or when the resting surface is a countertop/shelf in an ambulance or on a gurney.

### SUMMARY

In accordance with the present principles, an emergency medical device includes a treatment device including a display face, a base including a bottom portion configured to rest on a resting surface, and a back portion configured to extend beyond a rear surface of the treatment device opposite the front portion. A rear structure is connected to the treatment device and extends beyond the rear surface of the treatment device wherein the base is configured to provide a first angle of the display face when the bottom portion is resting on the resting surface, and wherein the back portion and the rear structure provide a second angle of the display face when the back portion and the rear structure are resting on the resting surface.

Another emergency medical device includes a base coupled to a treatment device having a display face. The base includes a front portion, a bottom portion configured to rest on a resting surface and a back portion configured to extend beyond a rear surface of the treatment device opposite the front portion. A rear bracket is connected to the treatment device and extends beyond the rear surface of the treatment device. The base is configured so that the front portion has a different offset dimension from the treatment device than the back portion to provide a first angle of the display face when the bottom portion is resting on the resting surface. The back portion and the rear bracket provide a second angle of the display face when the back portion and the rear bracket are resting on the resting surface.

Yet another emergency medical device includes a treatment device having a touchscreen display positioned on a face thereof. A base is coupled to the treatment device and includes a front portion, a bottom portion configured to rest on a resting surface and a back portion configured to extend beyond a rear surface of the treatment device opposite the face of the treatment device. At least one rear bracket is connected to the treatment device and extends beyond the rear surface of the treatment device wherein the base is configured to provide at least two viewing positions for the touchscreen display such that the front portion has a different offset dimension from the treatment device than the back portion to provide a first angle of the touchscreen display when the bottom portion is resting on the resting surface, and wherein the back portion and the at least one rear bracket provide a second angle of the touchscreen display when the back portion and the at least one rear bracket are resting on the resting surface. A handle is disposed on the treatment device on a side opposite that of the base, the handle permitting a user to adjust the device between the at least two viewing positions.

A method for positioning an emergency medical device includes providing a treatment device with a display face, a base coupled to the treatment device, the base including a front portion, a bottom portion configured to rest on a resting surface and a back portion configured to extend beyond a rear surface of the treatment device opposite the front portion; at least one rear bracket connected to the treatment device and extending beyond the rear surface of the treatment device wherein the base is configured so that the front portion has a different offset dimension from the treatment device than the back portion to provide a first angle of the display face when the bottom portion is resting on the resting surface, and wherein the back portion and the at least one rear bracket provide a second angle of the display face when the back portion and the at least one rear bracket are resting on the resting surface; and changing positions of the device between the first and second angles relative to resting surfaces in accordance with an operator position.

These and other objects, features and advantages of the present disclosure will become apparent from the following detailed description of illustrative exemplary embodiments thereof, which is to be read in connection with the accompanying drawings.

### BRIEF DESCRIPTION OF DRAWINGS

This disclosure will present in detail the following description of preferred embodiments with reference to the following figures wherein:

FIG. 1 is a front view showing a defibrillator/monitor device with a base and a handle in accordance with one embodiment;

FIG. 2 is a side view showing the defibrillator/monitor device of FIG. 1 with the base in contact with a resting surface to provide a first preset angle in accordance with one embodiment;

FIG. 3 is a side view showing the defibrillator/monitor device of FIG. 2 with the base and a rear bracket in contact with a resting surface to provide a second preset angle in accordance with one embodiment;

FIG. 4 is a bottom view showing the defibrillator/monitor device of FIG. lwith a U-shaped base in accordance with one embodiment; and

FIG. 5 is a block/flow diagram showing a method for operating the defibrillator/monitor in different positions in accordance with illustrative exemplary embodiments.

### DETAILED DESCRIPTION OF EXEMPLARY EMBODIMENTS

In accordance with the present principles, monitors and/or defibrillators may be provided with a touchscreen interface. The operation of a touchscreen on a monitor/defibrillator in accordance with the present principles employs more than one angle of the screen relative to a resting surface to accommodate multiple postures of medics in different care settings. The monitor/defibrillator includes geometry with mechanical features that permit users to position the touchscreen at more than one angle. For example, one angle of the display may appropriately position the touchscreen for an operator kneeling on the ground or floor. This is particularly useful where medics and first responders provide care from a kneeling position at accident scenes where patients collapse. A second angle may be employed to position the touchscreen for a seated or standing operator. During transport, medics may sit or stand in the back of an ambulance while caring for the patient. A monitor/defibrillator with features for multi-angle positioning meets the needs of medics that operate the user interface from more than one position or posture.

In particularly useful embodiments, operators utilize the touch screens to interact with the devices using single-touch controls. Menus and soft keys are activated using a single touch by the user on the touch screen. Multi-touch controls may also be employed for zooming, paging, scrolling, and other display navigation functions accomplished by having the user “pinch and expand” or “touch and swipe” the touch screen. A graphical user interface in accordance with the present principles utilizing touch screen technology provides both single-touch and multi-touch controls. By providing preset stable angle positioning, touchscreen operations are easily carried out in multiple positions in an ergonomic manner (e.g., without back bending of the wrists, etc.)

Devices in accordance with the present principles have mechanical features and geometry to permit users to position a display at more than one angle to accommodate multiple postures of caregivers in different care settings. The design geometry and features are simple and passive with no mechanisms, latches or moving parts to malfunction. The mechanical design allows fast operation with one hand in time critical, urgent care settings.

It should be understood that the present invention will be described in terms of medical instruments; however, the teachings of the present invention are much broader and are applicable to any monitor device or defibrillator operated in different positions. In some exemplary embodiments, the present principles are employed in defibrillators/monitors used in hospitals or emergency vehicles, in homes, in public places, etc. The elements depicted in the FIGS. may be implemented in various combinations of hardware and software and provide functions which may be combined in a single element or multiple elements.

The functions of the various elements shown in the FIGS. can be provided through the use of dedicated hardware as well as hardware capable of executing software in association with appropriate software. When provided by a processor or controller within a treatment device, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and exemplary embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention.

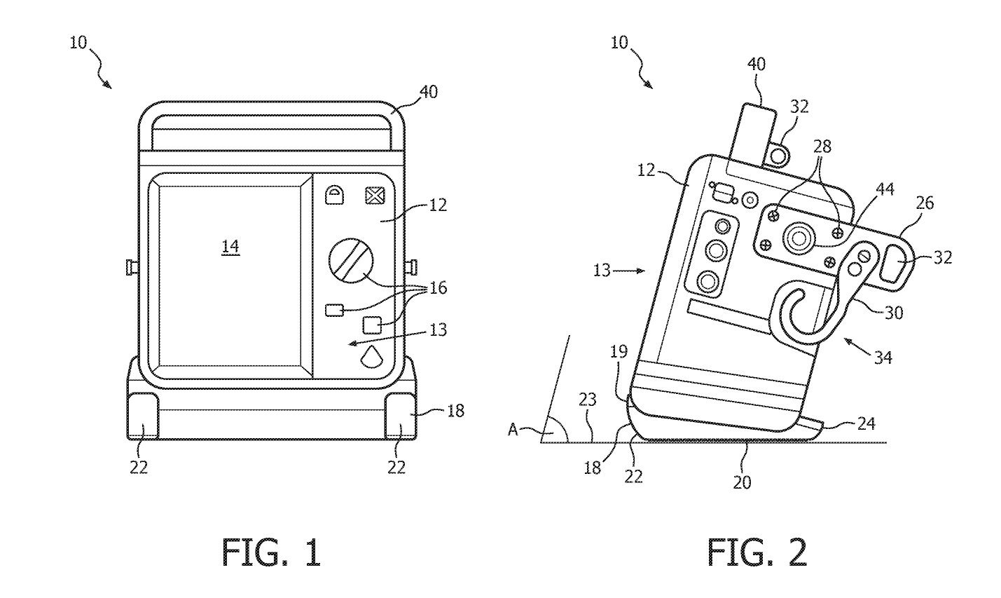

Referring now to the drawings in which like numerals represent the same or similar elements and initially to FIGS. 1 and 2, a diagram shows an emergency medical apparatus or device 10, which includes a treatment device 12, such as a defibrillator, monitor, etc. or combination thereof in accordance with one embodiment. Device 12 includes a plurality of controls 16 and a touch screen interactive display 14 on a display face 13. Apparatus 10 or device 12 includes one or more mechanical features that that are attachable externally to the device 12 or may be integrated into the device 12.

In one embodiment, the mechanical features may include a base 18 connectable to the device 12 below the display 14. The base 18 includes a front portion 22 that includes connection points 19 to a front of the device 12. The connection points 19 may include snap-fit mechanisms, screw holes, rivets, magnets, etc. or other connection mechanisms to hold the front portion 22 to the device 12. The base 18 may be permanently affixed to or temporarily mounted to the device 12. The base 18 provides a larger support height at the front portion 22 than a support height of a back portion 24. In this way, the base 18 supports device 12 in an upright configuration at an angle “A” preferably between about 45 degrees to less than about 90 degrees with respect to a resting surface 23. In one embodiment, the angle A is even more preferably between about 60 degrees and 80 degrees. A bottom portion 20 rests on the resting surface 23 and may include grips, treads or other mechanical features that prevent slippage, reduce or absorb shock, etc. The base 18 may include a combination of materials including rubber, plastics, metals, ceramics, etc. In one embodiment, at least the bottom portion 20 includes a rubber or rubberized surface.

In one exemplary embodiment, the mechanical design features on the base 18 of the device 12 position the touchscreen 14 to a 75° angle relative to a resting surface. The mechanical features or “feet” of the base 18 provide a stable base to support the entire weight of the device 12 from front to rear on each side and across a back edge or back portion 24 of the bottom portion 20. FIG. 2 shows a side view of the bottom portion 24 on the resting surface 23 to achieve the desired touchscreen angle of 75°. The bottom portion 20 may be textured to prevent slippage on smooth surfaces and is preferably made from durable, compliant material to help prevent damage from drop shock.

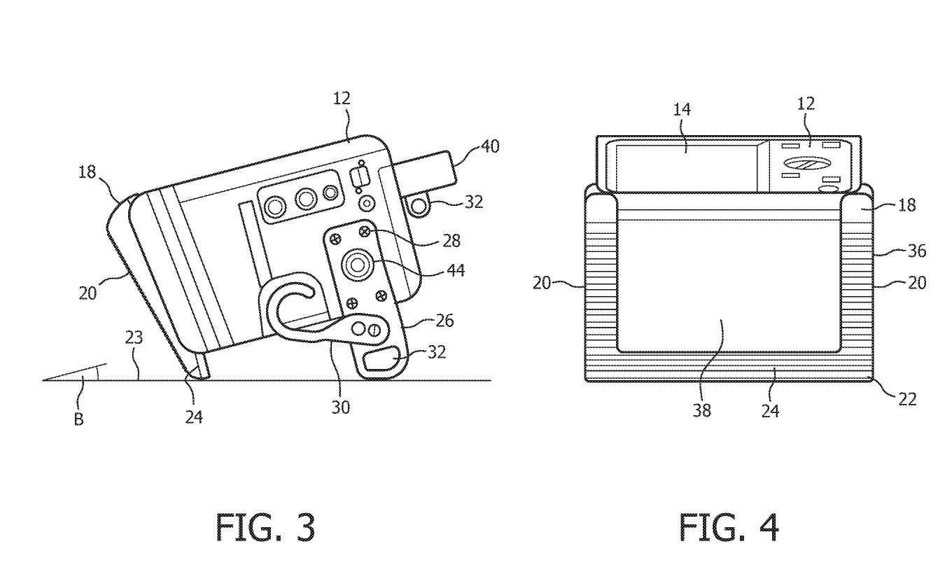

The position of the touch screen 14 in FIG. 2 provides an improved operational position while an operator is seated or standing. An angle A between about 60° and 90° is preferred in such positions. FIG. 2 also depicts a rear bracket 26 affixed to a side (or back surface) of the device 12. There may be a pair of rear brackets 26 one on each side of the device 12 that may be bolted, riveted, snapped or otherwise attached to the device 12. The rear brackets 26 may be connected to one another across the rear of the treatment device 12. The rear brackets 26 may take on any suitable configuration or structure. The rear brackets 26 may include a shock absorbing, weight bearing material to permit the device to be rotated backward to support the device 12 in a position shown in FIG. 3.

Referring to FIG. 3, the device 12 is depicted in a second position in which the rear brackets 26 and the back portion 24 are in contact with a resting surface. An angle position B for touch screen operation while an operator is in a kneeling position is preferably between about 0° to about 30°. In one embodiment, mechanical design features (the rear brackets 26 and the back portion 24) at the rear of the device 12, position the touchscreen at a 15° angle relative to the resting surface. The first position described in FIG. 2 is provided based upon a shape of the base 18 whereas the second position in FIG. 3 is provided based upon the rear brackets 26 and the back portion 24 of the base 18. The same bottom surface features (24) (“feet”) described above that extend slightly beyond the rear edge of the device 12 are advantageously employed. The rear brackets 26 also provide attachment points for rail hooks 30, shoulder strap eyes 32, etc. These can be located at multiple locations. A space 34 can be provided for rear storage or a storage pouch. Together, the rear brackets 26 of the back portion 24 form a stable support to achieve the desired 15°0 angle (or other angle) for operating the device from a kneeling or squatting position of the operator.

The base 18 and/or brackets 26 may include materials, such as plastic, metal, elastomers, etc. and fabrication methods may include, e.g., injection molding, machining, etc. A top handle 40 allows medics or operators to use a single hand to grasp the device top and push back the device to the low angle position of operation (0° to 30°) while kneeling. Alternatively, by grasping the handle 40 and pulling forward, the medic can return the device 12 to the high angle position of operation (60° to 90°) for the operation while seated/standing.

In one embodiment, the rear brackets 26 may be rotatably adjusted to change the second position angle B. In this embodiment, screws 28 may be removed or not included and a pivot 44 may be adjusted to determine a new angle for the second position. In other exemplary embodiments, the base 18 may be configured to provide an adjustment of the first position using a thumb screw or other adjustment mechanism to change the angle A. In yet other exemplary embodiments, other brackets or adjustments may be available to provide additional adjustment or to provide more than two set positions for the apparatus 10, for example, further extending or retracting brackets 26 or the back portion 24 of the base 18.

Referring to FIG. 4, a bottom view of the apparatus 10 is illustratively shown in accordance with one embodiment. In this embodiment, the bottom portion 20 of the base 18 includes a “U” shape following along sides and back of the apparatus 10. The bottom portion 20 includes grip features, treads 36 or textures to prevent slippage, etc. In some exemplary embodiments, a space 38 can include other structures or base features that can be integrated in or connect to the device 12 and provide support, dampening or additional structures.

In some exemplary embodiments, the base, back portion, rear brackets and/or other portions and aspects of the apparatus can be geometrically structured with unique mechanical features for secure placement of the apparatus in an ambulance (e.g., on an internal wall and elevated off the floor of an ambulance) or to a hospital bed (e.g., to the bed's base frame and/or rail(s)). Adjusting and/or locking mechanisms, such as those described herein above, for example, can be structured and integrated on or in to the apparatus and/or any portion thereof to be used to removably attach, secure and adjust the apparatus and display for multi-angle positioning for user-preferred viewing and access of the display.

It is also possible for the base, back portion, rear brackets and/or other portions and aspects of the apparatus to be geometrically structured and configured to be independently or asymmetrically adjustable from one another to provide stability on uneven surfaces (inside or outside). Independently telescoping feet can also be integrated and used to provide stability on uneven surfaces and providing user-preferred viewing and access of the display. Telescoping feet and other portions and features of the apparatus can also be extended out from the apparatus and/or in opposite directions from one another to opposingly press against walls or other surfaces to secure the apparatus in place.

While the present principles are described in terms of a monitor/defibrillator, two types of monitor defibrillators, in particular, can benefit from multi-angle positioning as described herein. These include in-hospital monitor defibrillators (used by hospital personnel) and pre-hospital monitor defibrillators (used by emergency medical services (EMS), public safety, military personnel, etc.). Other medical devices used in care settings that call for different operating postures may also benefit from the present principles.

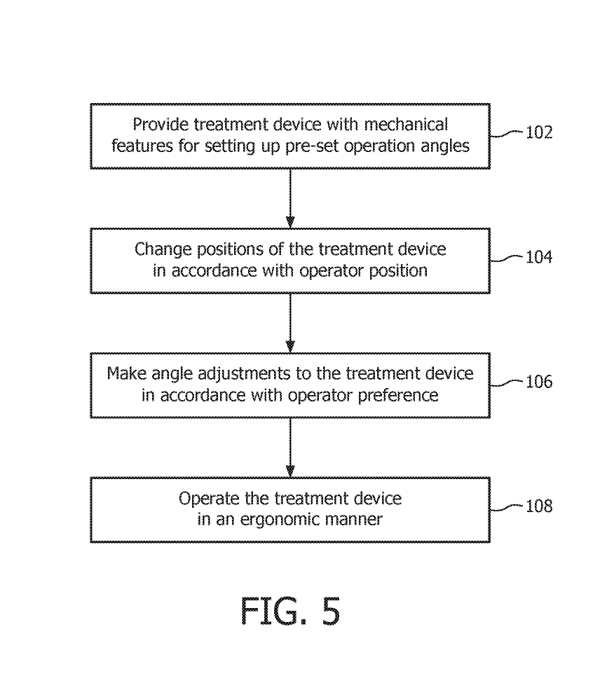

Referring to FIG. 5, a method for positioning an emergency medical device is illustratively shown in accordance with the present principles. In block 102, a treatment device with a display face is provided. The treatment device preferably includes a touchscreen display on the display face, and mechanical features for setting stable preset angle positions for the display. The treatment device may be integrated with or be retrofitted with a base and at least one rear bracket. The base is coupled (formed or connected) to the treatment device and includes a front portion, a bottom portion configured to rest on a resting surface and a back portion configured to extend beyond a rear surface of the treatment device opposite the front portion. The rear bracket or brackets is/are connected to the treatment device and extend beyond the rear surface of the treatment device. The base is configured so that the front portion has a different offset dimension from the treatment device than the back portion to provide a first angle of the display face when the bottom portion is resting on the resting surface. The back portion and the rear bracket(s) provide a second angle of the display face when the back portion and the at least one rear bracket are resting on the resting surface.

In block 104, the device can have its positions changed, e.g., between the first and second angles relative to a resting surface and in accordance with an operator position. The treatment device includes the touchscreen display positioned on the display face and the first angle and the second angle permit ergonomic touchscreen operation by a user in different positions. For example, at the first angle, e.g., between about 60 degrees and about 90 degrees with the resting surface, the operator may be sitting or standing. At the second angle between about 0 degrees and about 30 degrees with the resting surface, the operator may be kneeling or squatting. The device is advantageously tilted back or forward to provide a preset display angle that is optimized for the position of the operator.

The device may include more than two present angle positions by providing other mechanical features to support additional stable positions of the device. In block 106, adjustments may be made to the base and/or the rear brackets to adjust the first and/or second angles. This may be made based on operator preference or other considerations.

In block 108, the device is operated in an ergonomic manner using the touchscreen display. Since the touch screen display is appropriately angled based on the user's position, back bending of the wrist and other arm or wrist discomfort are avoided.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for monitor defibrillator with multi-angle positioning (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the exemplary embodiments disclosed herein as outlined by the appended claims. Having thus described the details and particularity required by the patent laws, is set forth in the appended claims.

What is claimed and desired protected by Letters Patent:

1. An emergency medical device, comprising:
a treatment device including:
a display face;
a base including a bottom portion configured to rest on a resting surface, and a back portion configured to extend beyond a rear surface of the treatment device opposite the front portion; and
a rear structure connected to the treatment device and extending beyond the rear surface of the treatment device;
wherein the base is configured to provide a first angle of the display face when the bottom portion is resting on the resting surface, and wherein the back portion and the rear structure provide a second angle of the display face when the back portion and the rear structure are resting on the resting surface.

2. The device as recited in claim 1, wherein the treatment device includes a touchscreen display positioned on the display face of the treatment device.

3. The device as recited in claim 1, wherein the bottom portion includes treads structured and configured to prevent slippage relative to the resting surface.

4. The device as recited in claim 1, wherein the base includes a U-shaped bottom portion extending along sides and the rear surface of the treatment device.

5. The device as recited in claim 1, wherein the rear structure includes at least one rear bracket affixed to at least one side of the treatment device.

6. The device as recited in claim 5, wherein the at least one rear bracket includes a support accessory including one or more of: a support hook and a support eye.

7. The device as recited in claim 1, wherein the first angle is between about 60 degrees and about 90degrees with the resting surface.

8. The device as recited in claim 1, wherein the second angle is between about 0 degrees and about 30degrees with the resting surface.

9. The device as recited in claim 1, wherein the treatment device includes at least one of a monitor, a defibrillator or a combination thereof.

10. An emergency medical device, comprising:
a treatment device having a touchscreen display positioned on a face thereof;
a base coupled to the treatment device and including:
a front portion;
a bottom portion configured to rest on a resting surface; and
a back portion configured to extend beyond a rear surface of the treatment device opposite the face of the treatment device;
at least one rear bracket connected to the treatment device and extending beyond the rear surface of the treatment device;
wherein the base is structured and configured to provide at least two viewing positions for the touchscreen display such that the front portion has a different offset dimension from the treatment device than the back portion to provide a first angle of the touchscreen display when the bottom portion is resting on the resting surface, and wherein the back portion and the at least one rear bracket provide a second angle of the touchscreen display when the back portion and the at least one rear bracket are resting on the resting surface; and
a handle disposed on the treatment device on a side opposite that of the base, the handle permitting a user to adjust the device between the at least two viewing positions.

11. The device as recited in claim 10, wherein the bottom portion includes treads structured and configured to prevent slippage relative to the resting surface.

12. The device as recited in claim 10, wherein the base includes a U-shaped bottom portion extending along sides and the rear surface of the treatment device.

13. The device as recited in claim 10, wherein the at least one rear bracket includes rear brackets affixed to opposite sides of the treatment device.

14. The device as recited in claim 10, wherein the at least one rear bracket includes a support accessory including one or more of: a support hook and a support eye.

15. The device as recited in claim 10, wherein a first position forms an angle of between about 60 degrees and about 90 degrees with the resting surface.

16. The device as recited in claim 10, wherein a second position forms an angle of between about 0 degrees and about 30 degrees with the resting surface.

17. The device as recited in claim 10, wherein the handle includes a support eye.

18. The device as recited in claim 10, wherein the treatment device includes one of a monitor, a defibrillator or a combination thereof.

19. A method for positioning an emergency medical device, comprising:
providing a treatment device with a display face, a base coupled or integrated with the treatment device, the base including a front portion, a bottom portion configured to rest on a resting surface and a back portion configured to extend beyond a rear surface of the treatment device opposite the front portion; at least one rear structure connected to the treatment device and extending beyond the rear surface of the treatment device wherein the base is configured so that the front portion has a different offset dimension from the treatment device than the back portion to provide a first angle of the display face when the bottom portion is resting on the resting surface, and wherein the back portion and the at least one rear structure provide a second angle of the display face when the back portion and the at least one rear structure are resting on the resting surface; and
changing positions of the device between the first and second angles relative to resting surfaces in accordance with an operator position.

20. The method as recited in claim 19, wherein the treatment device includes a touchscreen display positioned on the display face and the first angle and the second angle permit ergonomic touchscreen operation by a user in different positions.
